(19) United States
(12) Patent Application Publication
(10) Pub. No.: US 2017/0273666 A1
(43) Pub. Date: Sep. 28, 2017

(54) METHOD FOR STORING ULTRASONIC SCAN IMAGE AND ULTRASONIC DEVICE

(71) Applicants: Jiajiu YANG, Wuxi, Jiangsu (CN); Dongqing CHEN, Wauwatosa, WI (US); Yincheng LU, Wuxi, Jiangsu (CN); Wen I an MAO, Wuxi, Jiangsu (CN); Menachem HALMANN, Wauwatosa, WI (US); GENERAL ELECTRIC COMPANY, Schenectady, NY (US)

(72) Inventors: Jiajiu YANG, Wuxi, Jiangsu (CN); Dongqing CHEN, Wauwatosa, WI (US); Yincheng LU, Wuxi, Jiangsu (CN); Wenlan MAO, Wuxi, Jiangsu (CN); Menachem HALMANN, Wauwatosa, WI (US)

(21) Appl. No.: 15/514,000

(22) PCT Filed: Sep. 24, 2014
(86) PCT No.: PCT/CN2014/087266

§ 371 Date: Mar. 24, 2017

(51) Int. Cl. A61B 8/08

(52) U.S. Cl. CPC A61B 8/5215

(57) ABSTRACT

The present invention provides a method for storing an ultrasonic scan image and an ultrasonic device. The ultrasonic device may include: an image acquiring unit configured to scan a target object to obtain an image; a buffer unit configured to store the obtained image; a processing unit configured to compute a similarity of a frame of the image; and a storage unit, wherein when it is determined that the computed similarity of the frame is less than a threshold, the processing unit stores frames with a similarity equal to or greater than the threshold previous to the frame in the storage unit. Therefore, the image may be stored automatically based upon the similarity information of the frame of the ultrasonic scanning image.

### FIELD

The present invention relates to a method for storing an ultrasonic scan image and an ultrasonic device.

### BACKGROUND

An ultrasonic device usually includes an image acquiring unit for scanning a target object (e.g., a patient) to obtain an image, a display unit for displaying the image of the target object, and a storage unit for storing the obtained image.

When an operator scans the target object by operating the ultrasonic device, the operator firstly holds and moves the image acquiring unit (e.g., an ultrasonic probe), and simultaneously observes the image displayed on the display unit to determine whether the position of the image acquiring unit is at an area of interest. When it is determined that the image acquiring unit is located at the area of interest, the operator needs to manually operate the ultrasonic device, for example, to press a key on a front panel of the ultrasonic device such that the ultrasonic device starts to store the image, or stops a sweep and performs an analytical computation. During an examination for one patient, the operator needs to frequently move an arm to operate the ultrasonic device, memorize the image, or stop the sweep. In this way, the efficiency is decreased, and the image acquiring unit is likely to deviate from the area of interest, causing the operator to have to locate the image acquiring unit again, thus the stored images include undesired image, e.g., an image of an area of non-interest. Particularly at the time of guiding a puncture, since the operator is using both hands, operations from other people are required to complete such actions.

### SUMMARY

The object of exemplary embodiments of the present invention is to overcome the above and/or other defects in the prior art. Therefore, the exemplary embodiments of the present invention provide a method for automatically storing an ultrasonic scan image and an ultrasonic device.

According to an exemplary embodiment, a method for storing an ultrasonic scan image may include: computing a similarity of a frame of the image; determining whether the computed similarity of the frame is less that a threshold; and storing frames with similarities greater than or equal to the threshold previous to the frame when it is determined that the computed similarity of the frame is less than the threshold.

According to another exemplary embodiment, an ultrasonic device may include: means for computing a similarity of a frame of an image; means for determining whether the computed similarity of the frame is less than a threshold; and means for storing frames with similarities greater than or equal to the threshold previous to the frame when it is determined that the computed similarity of the frame is less than the threshold.

According to another exemplary embodiment, an ultrasonic device may include: an image acquiring unit configured to scan a target object to obtain an image; a buffer unit configured to store the obtained image; a processing unit configured to compute a similarity of a frame of the image; and a storage unit, wherein when it is determined that the computed similarity of the frame is less than a threshold, the processing unit stores frames with similarities greater than or equal to the threshold previous to the frame in the storage unit.

Other features and aspects will become apparent from the following detailed description, the figures and the claims.

### BRIEF DESCRIPTION OF THE DRAWINGS

The present invention can be understood better in light of the following description of exemplary embodiments of the present invention with reference to the accompanying drawings, in which:

FIG. 1 is a schematic flow chart illustrating a method for storing an ultrasonic scan image according an exemplary embodiment;

FIG. 2 is a schematic flow chart illustrating the steps of computing a similarity of a frame according to an exemplary embodiment;

FIG. 3 is a schematic flow chart illustrating the steps of storing according to an exemplary embodiment;

FIG. 4 is a schematic block diagram illustrating an ultrasonic device according to an exemplary embodiment.

### DETAILED DESCRIPTION

Hereafter, a detailed description will be given for preferred embodiments of the present invention. It should be pointed out that in the detailed description of the embodiments, for simplicity and conciseness, it is impossible for the Description to describe all the features of the practical embodiments in details. It should be understood that in the process of a practical implementation of any embodiment, just as in the process of an engineering project or a designing project, in order to achieve a specific goal of the developer and in order to satisfy some system-related or business-related constraints, a variety of decisions will usually be made, which will also be varied from one embodiment to another. In addition, it can also be understood that although the effort made in such developing process may be complex and time-consuming, some variations such as design, manufacture and production on the basis of the technical contents disclosed in the disclosure are just customary technical means in the art for those of ordinary skilled in the art relating to the contents disclosed in the present invention, which should not be regarded as insufficient disclosure of the present invention.

Unless defined otherwise, all the technical or scientific terms used in the Claims and the Description should have the same meanings as commonly understood by one of ordinary skilled in the art to which the present invention belongs. The terms “first”, “second” and the like in the Description and the Claims of the present application for invention do not mean any sequential order, number or importance, but are only used for distinguishing different components. The terms “a”, “an” and the like do not denote a limitation of quantity, but denote the existence of at least one. The terms “comprises”, “comprising”, “includes”, “including” and the like mean that the element or object in front of the “comprises”, “comprising”, “includes” and “including” encompasses the elements or objects and their equivalents illustrated following the “comprises”, “comprising”, “includes” and “including”, but do not exclude other elements or objects. The term “coupled”, “connected” or the like is not limited to being connected physically or mechanically, nor limited to being connected directly or indirectly.

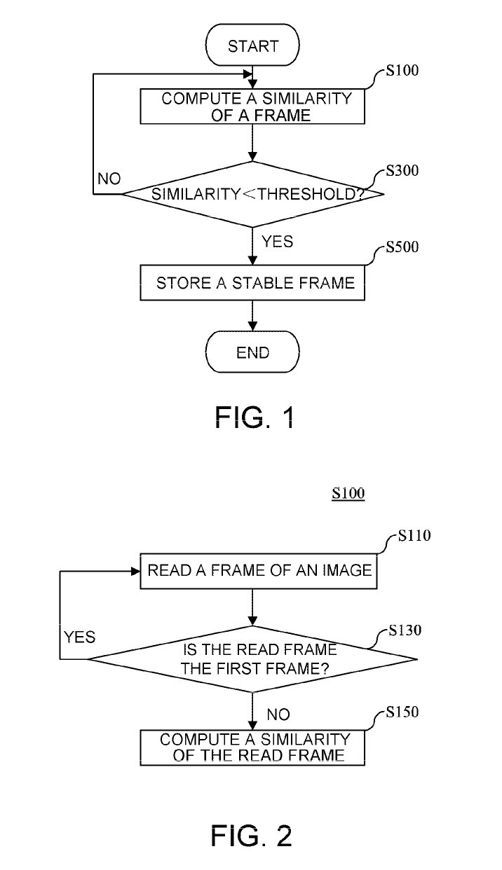

FIG. 1 is a schematic flow chart illustrating a method for storing an ultrasonic scan image according to an exemplary embodiment.

As shown in FIG. 1, firstly, in step S100, a similarity of a frame of an ultrasonic scan image may be computed. Herein, the image may be obtained in real time by performing an ultrasonic scanning on a target object (e.g., a patient). Thus, a similarity of the current frame may be computed. In other words, the method according to the exemplary embodiment may be carried out in real time while the ultrasonic scanning is performed.

FIG. 2 is a schematic flow chart illustrating the steps of computing a similarity of a frame according to an exemplary embodiment.

As shown in FIG. 2, firstly, a frame of an image, e.g., the current frame may be read (S110). Then, it may be determined whether the read frame is the first frame (S130). Herein, it may be determined whether the read frame is the first frame based upon, e.g., information indicative of the time (e.g., timestamp) at which the frame is obtained included in the read frame. When it is determined that the read frame is not the first frame (S130: NO), a similarity of the read frame to the previous frame may be computed (S150). On the contrary, when it is determined that the read frame is the first frame (S130: YES), the flow may return to step S110 to continue to read a frame of the image, e.g., read the second frame of the image.

According to one exemplary embodiment, the similarity of the read frame to the previous frame may be computed by the following equation (1):

$$C ( i , j ) = {\sum }_{u = 0}^{{M}_{a} - 1} {\sum }_{v = 0}^{{N}_{a} - 1} ( A ( u , v ) B ( u + i , v + j ) ) \qquad ( 1 )$$

Wherein the previous frame includes Mₐ×Nₐ pixels, the read frame includes M_{b}×N_{b} pixels, A(u,v) is a gray value of a pixel (u,v) in the previous frame, B(u+i,V+j) is a gray value of a pixel (u+i,v+j) of the current frame, 0 [symbol] i≦Mₐ+M_{b}−1, 0 [symbol] j [symbol] Nₐ+N_{b}−1. In this case, a maximum among the C(i,j) obtained by computing using equation (1) (or a maximum among the values obtained by normalizing the obtained C(i,j)) may be selected as the similarity of the previous frame of image to the read frame.

According to another exemplary embodiment, the similarity may be computed based upon a mean square deviation of the read frame with the previous frame. Specifically, H may be computed by the following equation (2).

H=1−Σᵢ₌₁^{M} Σⱼ₌₁^{N}(A_{i,j}−B_{i,j})² (2)

Wherein both the previous frame and the read frame include M×N pixels, A_{i,j} is a gray value of a pixel (i,j) in the previous frame, B_{i,j} is a gray value of a pixel (i,j) in the read frame, 1 [symbol] i [symbol] M, 1 [symbol] j [symbol] N.

According to yet another exemplary embodiment, the similarity may be computed based upon a normalized correlation value between the read frame and the previous frame. In other words, the normalized correlation value between the read frame and the previous frame may be employed as an index to measure the magnitude of the similarity. Specifically, the similarity may be computed by the following equation (3):

$$H = \frac{{\sum }_{i = 1}^{M} {\sum }_{j = 1}^{N} ( {A}_{i , j} - \overline{A} ) ( {B}_{i , j} - \overline{B} )}{\sqrt{{\sum }_{i = 1}^{M} {\sum }_{j = 1}^{N} {( {A}_{i , j} - \overline{A} )}^{2}} \sqrt{{\sum }_{i = 1}^{M} {\sum }_{j = 1}^{N} {( {B}_{i , j} - \overline{B} )}^{2}}} \qquad ( 3 )$$

Wherein both the previous frame and the read frame include M×N pixels, A_{i,j} is a gray value of a pixel (i,j) in the previous frame, B_{i,j} is a gray value of a pixel (i,j) in the read frame, 1 [symbol] [symbol] M, 1 [symbol] j [symbol] N, Ā and B̅ are an average gray value of the pixels of the previous frame and the read frame respectively.

The advantage of employing the normalized correlation value to measure the similarity is that it will not be affected by a linear transformation of the gray value of the image.

However, the method for computing a similarity of a frame is not limited thereto, a plurality of computing methods have been described in CN patent application No. 201410395924.6, which is incorporated herein by reference in its entirety.

Returning to refer to FIG. 1, after the similarity of the frame has been computed, it may be determined whether the computed similarity is less than a threshold (S330). Herein, the threshold may be preset. For example, different thresholds may be set according to different parts of the target object. In addition, a user may modify the threshold according to the requirements. The greater the similarity is, the more similar the frame is to the previous frame. Therefore, a frame with a similarity less than the threshold may have a larger difference compared with the previous frame, and thus it may be determined that currently the operator is moving the image acquiring unit (e.g., an ultrasonic probe) of the ultrasonic device in a relatively large magnitude. This means that the operator may perhaps be performing a location operation of the image acquiring unit. On the other hand, the frame with a similarity greater than or equal to the threshold may have a small difference compared with the previous frame, and thus it may be determined that currently the operator is sweeping statically or moving the image acquiring unit (e.g., the ultrasonic probe) of the ultrasonic device in a relatively small magnitude. This means that the operator may perhaps be scanning an area of interest of the target object. In other words, it may be judged whether the ultrasonic scanning operation for obtaining the frame is the location operation of the image acquiring unit or the scanning operation on the area of interest based upon the similarity information of the frame of the ultrasonic scan image. In the following, the frame with a similarity greater than or equal to the threshold may be called as a stable frame.

Therefore, when it is determined that the computed similarity of the frame is less than the threshold (S300: YES), the stable frames with similarities greater than or equal to the threshold previous to the frame may be stored (S500). On the other hand, when it is determined that the computed similarity of the frame is greater than or equal to the threshold (S300: NO), or when there is no stable frame with a similarity greater than or equal to the threshold in the frames previous to the frame, the flow may return to step S100 to compute a similarity of another frame (e.g., the next frame) of the image.

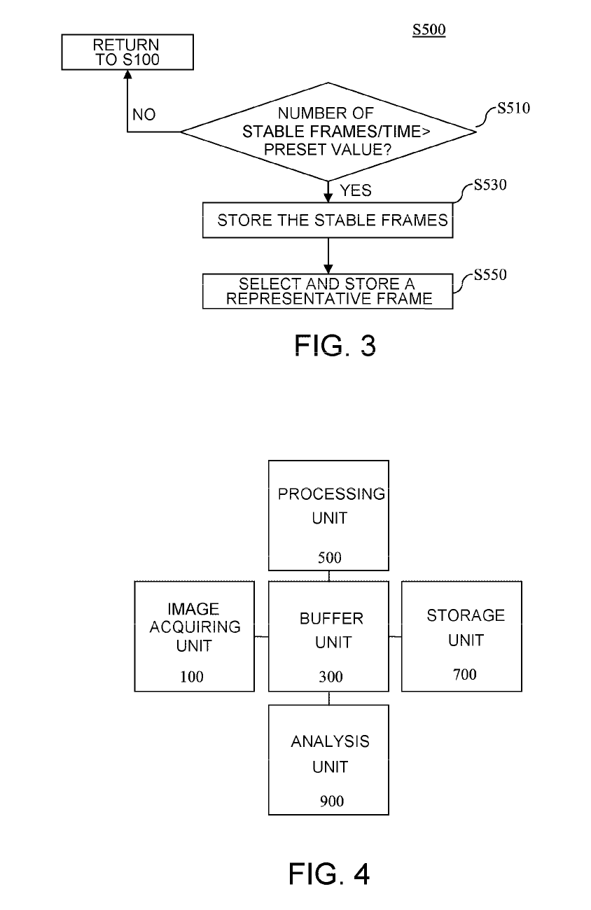

FIG. 3 is a schematic flow chart illustrating the steps of storing according to an exemplary embodiment.

As shown in FIG. 3, firstly, it may be determined whether the number and/or the time of the consecutive stable frames previous to the frame with a similarity less than the threshold is greater than a preset value (S510). Herein, the preset value may be set in advance. For example, different preset values may be set according to different parts of the target object and/or an operation habit of the operator. When determining that at least one of the number and the time length of the consecutive stable frames is greater than the preset value (S510: YES), it may indicate that the operator may at that time scan an area of interest, and accordingly the consecutive stable frames may be stored (S530). For example, the consecutive stable frames may be stored as a video file. Moreover, a representative frame may also be selected among the consecutive frames and stored (S550). The representative frame may be selected automatically or manually. For example, the representative frame may be the last frame (or the first frame) in said consecutive frames, or a frame capable of representing a part of the target object displayed by said consecutive frames, for instance, the representative frame may be a frame showing the most blood flow areas.

On the other hand, when it is determined that at least one of the number and the time length of the consecutive stable frames is less than or equal to the preset value (S510: NO), it may indicate that the operator may at that time be distinguishing whether the area being scanned is an area of interest, and accordingly storing may not be performed and the flow returns to step S100.

Therefore, according to the exemplary embodiments, it may be judged whether the ultrasonic scanning operation at the time of obtaining the frame is a location operation of the image acquiring unit or a scanning operation on an area of interest based upon the similarity information of the frame of the ultrasonic scan image, and the image of the area of interest may be stored automatically in the case that a manual operation is not necessary, thus the ultrasonic scanning operation is simplified and the time of the ultrasonic scanning is shortened.

FIG. 4 is a schematic block diagram illustrating an ultrasonic device according to an exemplary embodiment. The ultrasonic device according to the present exemplary embodiment may carry out the methods as described above with reference to FIGS. 1-3. Therefore, the repetitive description for the same or similar elements or features will be omitted.

As shown in FIG. 4, the ultrasonic device according to the exemplary embodiment may include an image acquiring unit 100, a buffer unit 300, a processing unit 500, and a storage unit 700.

The image acquiring unit 100 may scan a target object to obtain an image. For example, the image acquiring unit 100 may include an ultrasonic probe for scanning, an image processor for processing the signals obtained by the ultrasonic probe to obtain the image, etc.

The buffer unit 300 may, e.g., store the image obtained by the image acquiring unit 100 in real time. For example, the buffer unit 300 may include a volatile memory.

The processing unit 500 may compute a similarity of a frame of the image. For example, the processing unit 500 may read the frame of the image from the buffer unit 300, and may determine whether the read frame is the first frame. Herein, it may be determined whether the read frame is the first frame based upon, e.g., information indicative of the time (e.g., timestamp) at which the frame is obtained included in the read frame. When determining that the read frame is not the first frame, the processing unit 500 may compute a similarity of the read frame to the previous frame.

According to the present exemplary embodiment, the processing unit 500 may compute the similarity of the read frame to the previous frame by anyone of the above equations 1-3 or other algorithms.

Alternatively, after computing a similarity, the processing unit 500 may store the computed similarity in the buffer unit 300. For example, the processing unit 500 may store similarities of various frames except the first frame in the buffer unit 300 in the form of a loop-up table.

The processing unit 500 may determine whether the computed similarity of the frame is less than a threshold. Herein, the threshold may be preset. For example, different thresholds may be set according to different parts of the target object. In addition, a user may modify the threshold according to the requirements. The greater the similarity is, the more similar the frame is to the previous frame. Therefore, a frame with a similarity less than the threshold may have a larger difference compared with the previous frame, and thus it may be determined that the operator is currently moving the image acquiring unit (e.g., an ultrasonic probe) of the ultrasonic device in a relative large magnitude. This means that the operator may perhaps be performing a location operation of the image acquiring unit. On the other hand, the frame with a similarity greater than or equal to the threshold may have a small difference compared with the previous frame, and thus it may be determined that the operator is currently sweeping statically or moving the image acquiring unit (e.g., the ultrasonic probe) of the ultrasonic device in a relatively small magnitude. This means that the operator may perhaps be scanning an area of interest of the target object. In other words, it may be judged whether the ultrasonic scanning operation of obtaining the frame is a location operation of the image acquiring unit or a scanning operation on the area of interest based upon the similarity information of the frame of the ultrasonic scan image. In the following, the frame with a similarity greater than or equal to the threshold may be called as a stable frame.

When it is determined that the computed similarity of the frame is less than the threshold, the processing unit 500 may store the stable frames with similarities greater than or equal to the threshold previous to the frame in the storage unit 700. Herein, the storage unit 700 may include a non-volatile memory, e.g., a flash memory, a hard disk driver, a solid-state disk, an optical disk, etc.

Although not shown in the figure, the ultrasonic device may also include a notifying unit. The notifying unit may send a notification to the user when the processing unit stores said consecutive frames into the storage unit. For example, the notifying unit may display information including a text, an image, etc. on a display for displaying the scanned image of the ultrasonic device, and/or may provide audio information, so as to notify the user (e.g., the operator) of being performing an automatic storing operation.

In one exemplary embodiment, the processing unit 500 may determine whether at least one of the number and the time length of the consecutive stable frames previous to the frame with a similarity less than the threshold is greater than a preset value. Herein, the preset value may be set in advance. For example, different preset values may be set according to different parts of the target object and/or an operation habit of the operator. When the processing unit 500 determines that at least one of the number and the time length of the consecutive stable frames is greater than the preset value, it may indicate that the operator may at that time scan an area of interest. Accordingly, the processing unit 500 may store said consecutive stable frames into the storage unit 700. For example, the processing unit 500 may store said consecutive stable frames as a video file. Moreover, the processing unit 500 may also select a representative frame among said consecutive frames and store the representative frame in the storage unit 700. The processing unit 500 may select the representative frame automatically. For example, the representative frame may be the last frame (or the first frame) in said consecutive frames, or a frame capable of representing a part of the target object displayed by said consecutive frames, for instance, the representative frame may be a frame showing the most blood flow regions.

In an alternative exemplary embodiment, the ultrasonic device may also include an analysis unit 900, as shown in FIG. 4. The analysis unit 900 may analyze the stored consecutive stable frames. The analysis unit 900 may perform various analyzing operations according to the presetting of the operator, for example, the analysis unit 900 may recognize various parts of the target object shown in the stable frames, or may compute the trend of change of the stable frames, etc.

Moreover, the analysis unit 900 may choose whether to discard the stored consecutive stable frames from the buffer unit 300 based upon the analysis result. In this way, the storage space of the buffer unit 300 may be released to optimize the use efficiency of the buffer unit 300. For example, when it is determined that the analysis result cannot reach the desire preset by the operator, the analysis unit 900 may discard the stored consecutive stable frames from the buffer unit 300. On the other hand, when it is determined that the analysis result may reach the desire preset by the operator, the analysis unit 900 may hold the stored consecutive stable frames in the buffer unit 300, and/or may stop the sweeping operation, so that the operator can directly read the stored consecutive stable frames from the buffer unit 300 at the time of manually analyzing the stored consecutive stable frames, such that an immediate analysis thereof may be allowed. In this way, the efficiency of the analyzing operation may be improved.

Although the analysis unit 900 and the processing unit 500 are shown as separate elements in FIG. 4, the exemplary embodiment is not limited thereto, for example, the analysis unit 900 and the processing unit 500 may be implemented as a single element, e.g., a microprocessor, a central processing unit (CPU), etc.

According to the exemplary embodiments, the ultrasonic device may recognize the ultrasonic scanning operation at the time of obtaining the frame based upon the similarity information of the frame of the ultrasonic scan image, and may automatically store the image of the area of interest in the case that a manual operation is not necessary, thus the ultrasonic scanning operation is simplified and the time of the ultrasonic scanning is shortened. Moreover, the ultrasonic device may also automatically and selectively release the storage space of the buffer unit such that the use efficiency of the buffer unit is optimized.

Some exemplary embodiments have been described in the above. However, it should be understood that various modifications may be made thereto. For example, if the described techniques are carried out in different orders, and/or if the components in the described system, architecture, device or circuit are combined in different ways and/or replaced or supplemented by additional components or equivalents thereof, proper results can still be achieved. Accordingly, other embodiments are also falling within the protection scope of the claims.

What is claimed is:

1. A method for storing an ultrasonic scan image, said method comprising:
computing a similarity of a frame of the image;
determining whether the computed similarity of the frame is less that a threshold; and
storing frames with similarities equal to or greater than the threshold previous to the frame when it is determined that the computed similarity of the frame is less than the threshold.

2. The method according to claim 1, wherein the step of computing a similarity of a frame comprises:
reading the frame of the image;
determining whether the read frame is the first frame; and
computing a similarity of the read frame to the previous frame when it is determined that the read frame is not the first frame.

3. The method according to claim 1, wherein the step of storing comprises:
determining whether at least one of a number and a time length of consecutive frames with similarities greater than or equal to the threshold previous to the frame with a similarity less than the threshold is greater than a preset value; and
storing said consecutive frames when it is determined that at least one of the number and the time length of said consecutive frames is greater than the preset value.

4. The method according to claim 3, wherein the step of storing said consecutive frames comprises:
storing said consecutive frames as a video.

5. The method according to claim 4, wherein the step of storing said consecutive frames further comprises:
selecting a representative frame among said consecutive frames; and
storing the selected representative frame.

6. An ultrasonic device, comprising:
means for computing a similarity of a frame of an image;
means for determining whether the computed similarity of the frame is less than a threshold; and
means for storing frames with similarities greater than or equal to the threshold previous to the frame when it is determined that the computed similarity of the frame is less than the threshold.

7. The ultrasonic device according to claim 6, wherein the means for computing a similarity of a frame of an image comprises:
means for reading the frame of the image;
means for determining whether the read frame is the first frame; and
means for computing a similarity of the read frame to the previous frame when it is determined that the read frame is not the first frame.

8. The ultrasonic device according to claim 6, wherein the means for storing comprises:
means for determining whether at least one of a number and a time length of consecutive frames with similarities greater than or equal to the threshold previous to the frame with a similarity less than the threshold is greater than a preset value; and
means for storing said consecutive frames when it is determined that at least one of the number and the time length of said consecutive frames is greater than the preset value.

9. The ultrasonic device according to claim 8, wherein the means for storing said consecutive frames comprises:
means for storing said consecutive frames as a video.

10. The ultrasonic device according to claim 9, wherein the means for storing said consecutive frames comprises:
means for selecting a representative frame among said consecutive frames; and
means for storing the selected representative frame.

11. An ultrasonic device, comprising:
an image acquiring unit configured to scan a target object to obtain an image;
a buffer unit configured to store the obtained image;
a processing unit configured to compute a similarity of a frame of the image; and
a storage unit, wherein the processing unit stores frames with similarities greater than or equal to the threshold previous to the frame in the storage unit when it is determined that the computed similarity of the frame is less than a threshold.

12. The ultrasonic device according to claim 11, wherein the processing unit is configured to read the frame of the image from the buffer unit, and compute a similarity of the read frame to the previous frame when it is determined that the read frame is not the first frame.

13. The ultrasonic device according to claim 12, wherein the processing unit is further configured to store the computed similarity in the buffer unit.

14. The ultrasonic device according to claim 12, wherein the processing unit is further configured to store consecutive frames into the storage unit when it is determined that at least one of a number and a time length of said consecutive frames with similarities greater than or equal to the threshold previous to the frame with a similarity less than the threshold is greater than a preset value.

15. The ultrasonic device according to claim 14, wherein the processing unit is further configured to discard the consecutive frames from the buffer unit when determining that at least one of the number and the time length of the consecutive frames with similarities greater than or equal to the threshold previous to the frame with a similarity less than the threshold is less than or equal to the preset value.

16. The ultrasonic device according to claim 14, wherein the ultrasonic device further comprises:
a notifying unit configured to send a notification to a user when the processing unit stores said consecutive frames into the storage unit.

17. The ultrasonic device according to claim 14, wherein the processing unit is configured to store said consecutive frames as a video.

18. The ultrasonic device according to claim 17, wherein the processing unit is further configured to select a representative frame among said consecutive frames and store the selected representative frame in the storage unit.

19. The ultrasonic device according to claim 11, wherein the ultrasonic device further comprises:
an analysis unit configured to analyze the stored consecutive frames, and hold the stored consecutive frames in the buffer unit and stop a sweeping based upon an analysis result.
